(19) United States
(12) Patent Application Publication
(10) Pub. No.: US 2009/0198266 A1
(43) Pub. Date: Aug. 6, 2009

(54) DEVICES AND METHODS FOR ABDOMINAL SURGERY

(75) Inventor: James Cesare, New Hartford, NY (US)

Correspondence Address: JBM MEDICAL LLC, 206 WATERMAN STREET, PROVIDENCE, RI, 02906, US

(73) Assignee: JBM Medical LLC

(21) Appl. No.: 12/132,582

(22) Filed: Jun. 3, 2008

Related U.S. Application Data
(60) Provisional application No. 61/025,439, filed on Feb. 1, 2008.

(51) Int. Cl. A61M 29/02

(52) U.S. Cl. 606/192

(57) ABSTRACT

A method includes expanding a first balloon distally of a gastro-esophageal junction, and contacting the first balloon to a proximal portion of a stomach. The method can be used for abdominal surgery, such as, for example, bariatic surgery (e.g., gastric bypass surgery), anti-reflux surgery (e.g., fundoplication), and other esophageal-related surgeries (e.g., Heller myotomy, hiatal hernia repair, and paraesophageal hernia repair).

### RELATED APPLICATION

This application claims benefit of U.S. Provisional Patent Application Ser. No. 61/025439, titled “Devices and Method for Abdominal Surgery,” filed Feb. 1, 2008 and which is incorporated by reference herein.

### TECHNICAL FIELD

The invention relates to devices and methods for abdominal surgery, such as, for example, bariatic surgery.

### BACKGROUND

Being severely over-weight, or morbidly obese, can be a serious medical condition that can markedly decrease life expectancy. For example, morbid obesity has been associated with conditions such as, for example, hypertension, diabetes, cardiovascular disease, stroke, congestive heart failure, sleep apnea, liver disease, gallbladder disease, gastroesophageal reflux disease (GERD), various forms of cancer, multiple orthopedic problems, and pulmonary insufficiency. Various treatments are known for restricting food intake and/or nutrient absorption, thereby reducing obesity in patients.

One treatment for obesity is gastric bypass surgery. Briefly, in this surgery, the stomach is divided into two unequal portions: a small upper pouch and a large lower pouch. A segment of the small intestine is then brought from the lower abdomen and joined with the small upper pouch. This segment of the small intestine carries the food from the small upper pouch to the remainder of the intestines, where the food is digested. The large lower pouch remains generally intact and continues to secrete stomach juices flowing through the intestinal track. The goal of gastric bypass surgery is to reduce the volume of the space in the stomach receiving food. When the patient ingests food, the small upper pouch is filled which stretches the stomach wall and produces satiety. The patient typically eats at a slower rate and is satiated much more quickly with less food intake, which can result in a reduction in caloric intake and subsequent weight loss.

### SUMMARY

The invention relates to devices and methods for abdominal surgery. Examples of abdominal surgery include bariatic surgery (e.g., gastric bypass surgery), surgery to treat gastroesophageal reflux disease (GERD) (e.g., partial fundoplication and Nissen fundoplication), and other esophageal-related surgeries (e.g., Heller myotomy, hiatal hernia repair, and paraesophageal hernia repair).

In one aspect, the invention features a method including expanding a first balloon distally of a gastro-esophageal junction; and contacting the first balloon to a proximal portion of a stomach.

Embodiments may include one or more of the following features. The method further includes connecting wall portions of the stomach together around a portion of the expanded first balloon. The method further includes expanding a second balloon proximally of the proximal portion of the stomach. The method further includes surgically reducing reflux from the stomach. The method further includes reducing or repairing a diaphragmatic defect. The method further includes performing at least one step of a Heller myotomy procedure. The second balloon is capable of having an expanded length of approximately 10 to approximately 15 centimeters. The second balloon is capable of having a maximum expanded outer diameter of approximately 20 millimeters. The method further includes performing at least one additional step of a bariatic surgical procedure. The method further includes orally or nasally introducing the first balloon into the body. The first balloon is capable of having an expanded volume of approximately 20 to approximately 30 cubic centimeters. The first balloon is carried by a shaft having a diameter of approximately 12 to approximately 28 French. The first balloon is carried by a shaft having a passageway extending along a longitudinal axis, and the method further includes passing an instrument through the passageway, the instrument a pumping instrument, an irrigation instrument, a contrast agent delivery instrument, an endoscope, or a light source.

In another aspect, the invention features a method including expanding a first balloon in a stomach; and connecting wall portions of the stomach together around a portion of the expanded first balloon.

Embodiments may include one or more of the following features. The method further includes contacting the expanded first balloon to a proximal portion of the stomach. The method further includes expanding a second balloon proximally of the stomach. The method further includes orally or nasally introducing the first balloon into the body. The first balloon is capable of having an expanded volume of approximately 20 to approximately 30 cubic centimeters. The first balloon is capable of having a substantially spherical expanded volume. The first balloon is carried by a shaft having an outer diameter of approximately 24 to approximately 28 French. The method further includes performing at least one additional step of a bariatic surgical procedure.

In another aspect, the invention features a medical device including a shaft; a first balloon carried by the shaft, the first balloon being capable of having a substantially spherical expanded volume; and a second balloon carried by the shaft and positioned spaced from the first balloon.

Embodiments may include one or more of the following features. A proximal-most expandable portion of the first balloon and a distal-most expandable portion of the second balloon are spaced approximately 10 millimeters to approximately 50 millimeters apart. The second balloon is capable of having an expanded length of approximately 10 to approximately 15 centimeters. The second balloon is capable of having a maximum expanded outer diameter of approximately 20 millimeters. The first balloon is capable of having an expanded volume of approximately 20 to approximately 30 cubic centimeters. The shaft has an outer diameter of approximately 24 to approximately 28 French. The device has an overall length of approximately 20 centimeters to approximately 80 centimeters. The device is free of an expandable stent and a sheath covering the balloons. The shaft has a passageway extending along a longitudinal length of the shaft, the passageway being open at a distal tip of the shaft. The shaft has a distal tip spaced less than approximately 5 millimeters from an expandable portion of the first balloon.

In another aspect, the invention features a medical device including a shaft; a first balloon carried by the shaft, the first balloon being capable of having a substantially spherical expanded volume; and a second balloon carried by the shaft and positioned spaced from the first balloon, the second balloon being capable of having an expanded length of approximately 10 to approximately 15 centimeters.

Embodiments may include one or more of the following features. A proximal-most portion of the first balloon and a distal-most portion of the second balloon are spaced approximately 1 centimeter to approximately 2 centimeters apart. The second balloon is capable of having a maximum expanded outer diameter of approximately 20 millimeters. The first balloon is capable of having an expanded volume of approximately 20 to approximately 30 cubic centimeters. The shaft has an outer diameter of approximately 24 to approximately 28 French. The device has an overall length of approximately 20 centimeters to approximately 80 centimeters. The device is free of an expandable stent and a sheath covering the balloons. The shaft has a passageway extending along a longitudinal length of the shaft, the passageway being open at a distal tip of the shaft. The shaft has a distal tip spaced less than approximately 5 millimeters from an expandable portion of the first balloon.

Embodiments may include one or more of the following advantages.

The devices and methods can be used in abdominal surgical procedures, including laparoscopic procedures and open procedures. These procedures can be performed with enhanced accuracy, precision, and safety. For example, in a gastric bypass procedure, the visualization and ease of creating a consistent upper pouch can be standardized.

The devices are small in size, are not cumbersome, and do not require much time to use. As a result, use of the devices can carry a reduced or no risk for perforation or other complications that may be associated with other devices.

Surgical efficiency can be enhanced because the devices and methods are easy to use, do not require much time, and can be readily taught.

As used herein, “a gastro-esophageal junction”, sometimes referred to as “a GE junction”, is synonymous with “an esophago-gastric junction”.

Other aspects, features and advantages will be apparent from the description of the embodiments thereof and from the claims.

### BRIEF DESCRIPTION OF DRAWINGS

FIG. 1 is a schematic diagram of an embodiment of a medical device.

FIG. 2 is cross section taken along line 2-2 in FIG. 1.

FIG. 3 is a detailed view of area 3 in FIG. 1.

FIGS. 4A, 4B, 4C, and 4D are illustrations showing an embodiment of a bariatic surgical procedure.

FIGS. 5A, 5B, and 5C are illustrations showing an embodiment of a Nissen fundoplication or complete fundoplication surgical procedure.

FIG. 6 is an illustration showing an embodiment of a Heller myotomy surgical procedure.

FIG. 7 is an illustration showing an embodiment of a surgical procedure to repair a defect in a diaphragm.

FIG. 8 is a schematic diagram of an embodiment of a medical device.

### DETAILED DESCRIPTION

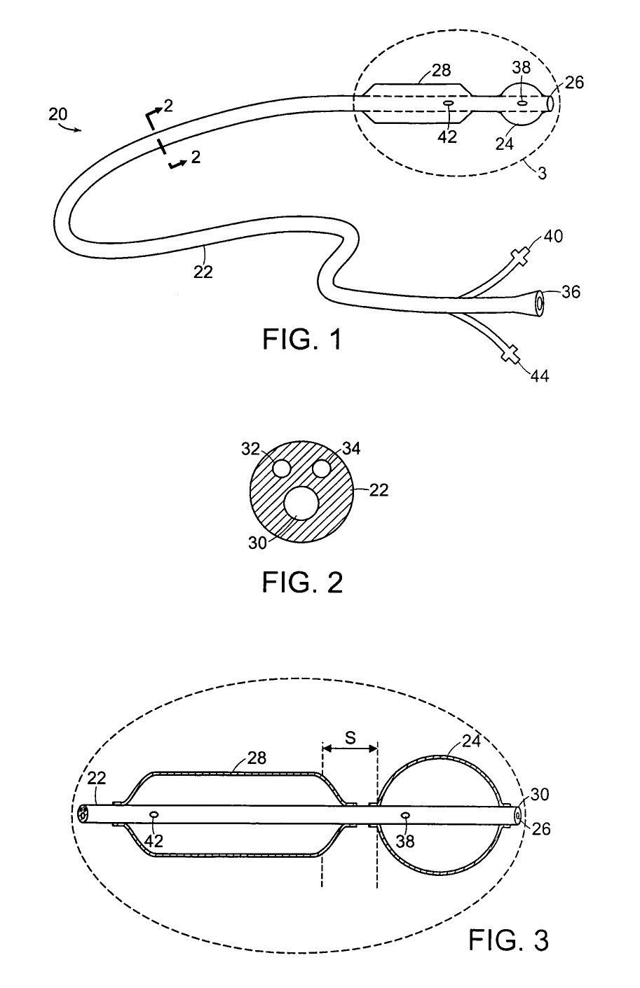

FIG. 1 shows a medical device 20 capable of being used in abdominal surgery. Device 20 includes a flexible shaft 22, a first expandable balloon 24 carried by the shaft near its distal end 26, and a second expandable balloon 28 carried by the shaft proximal to the first expandable balloon. Referring also to FIG. 2, shaft 22 has three lumens or passageways 30, 32, 34 extending along the longitudinal length of the shaft. Passageway 30 is open at distal end 26 and extends from the distal end to a proximal end 36 of device 20. Passageway 30 is sized to allow an instrument (such as an endoscope) to pass through the passageway from proximal end 36 to distal end 26. Passageway 32 and passageway 34 can be used independently to inflate and to deflate first balloon 24 and second balloon 28, respectively. Passageway 32 extends from an opening 38 that is in fluid communication with an interior volume of first balloon 24 to a proximal port 40, where a fluid can be introduced and removed from passageway 32 and the first balloon. Passageway 34 extends from an opening 42 that is in fluid communication with an interior volume of second balloon 28 to a proximal port 44, where a fluid can be introduced and removed from passageway 34 and the second balloon.

As described further below, first balloon 24 can be used to facilitate a bariatic surgical procedure, for example, to identify the gastro-esophageal (GE) junction and the proximal portion of the stomach, and to facilitate creation of an upper stomach pouch. As shown in FIG. 3, first balloon 24 is positioned near distal end 26, for example, such that the distance between the distal end and the closest expandable portion of the first balloon is less than or equal to approximately five centimeters (e.g., less than or equal to approximately two centimeters). Limiting the distance that shaft 22 extends distally from first balloon 24 allows the upper stomach pouch to be formed well-matched to the expanded first balloon, as described below. In some embodiments, shaft 22 does not extend distally beyond the distal-most expandable portion of first balloon 24. As shown, first balloon 24 is capable of being inflated to a fully expanded shape that is substantially spherical, although other shapes (e.g., prolate, oblate, polyhedral with or without facets) are possible. In some embodiments, first balloon 24 has a volume of approximately 20 to approximately 30 cubic centimeters when it is fully expanded.

Second balloon 28 can be used, for example, to dilate, to size, and/or to maintain the patency of the esophagus. Still referring to FIG. 3, second balloon 28 is positioned spaced from and proximal to first balloon 24. In some embodiments, balloons 24, 28 are spaced approximately 10 millimeters to approximately 50 millimeters apart, as measured from the proximal-most expandable portion of first balloon 24 and the distal-most expandable portion of second balloon 28 where the diameter is at its maximum (S, FIG. 3). The spacing (S) between balloons 24, 28 can correspond approximately to the length of the GE junction, which can help a surgeon locate the GE junction and properly dilate the esophagus, as described below. In some embodiments, second balloon 28, when fully expanded, has a maximum outer diameter of approximately 15 millimeters to approximately 25 millimeters (e.g., approximately 20 millimeters, or the size equivalent to a 60 French bougie). The length of the expandable portion of second balloon 28 capable of having the maximum outer diameter can range from approximately ten centimeters to approximately 15 centimeters. This length of second balloon 28 allows device 20 to be used in certain surgical procedures, such as a fundoplication procedure or a Heller myotomy procedure. The cross section of the expandable portion of second balloon 28 capable of having the maximum outer diameter can be substantially round (e.g., oval, elliptical, circular) or not round (e.g., regularly or irregularly polygonal).

Shaft 22 is an elongated member configured to be inserted nasally or orally into the esophagus. In some embodiments, shaft 22 has an outer diameter equivalent to approximately 12 French to approximately 28 French. Small diameter shafts are easier to insert into the patient and may be more comfortable for the patient, but large diameter shafts may allow larger instruments to be passed through passageway 30. The length of shaft 22, as measured from distal end 26 to proximal end 36, can range from approximately 20 centimeters to approximately 80 centimeters, but it is not so limited.

Device 20 can include (e.g., be formed of) any material capable of being of in the body for a surgical procedure. Examples of materials include biocompatible polymers, such as thermoplastics, elastomers and thermosets used for catheters, e.g., balloon catheters. Specific examples of materials include polyolefins, polyamides, such as nylon 12, nylon 11, nylon 6/12, nylon 6, and nylon 66, polyesters, polyethers, polyurethanes, polyureas, polyvinyls, polyacrylics, fluoropolymers, copolymers and block copolymers thereof, such as block copolymers of polyether and polyamide, e.g., Pebax®, EPDM, epichlorohydrin, nitrile butadiene elastomers, silicones, epoxies, isocyanates, and mixtures thereof Composites, such as those including a polymer and a second material (e.g., clays, nanoparticles, nanotubes), can also be used. The materials can be used by themselves or as combinations of two or more different materials (e.g., intimate blends, combinations having discrete portions with different compositions, such as multilayered balloons and/or shafts). Methods of making medical devices are described, for example, in U.S. Published Application Nos. 20050043712 and 20060184112.

Device 20 can include one or more materials selected to enhance its performance. For example, device 20 can include one or more lubricants (such as a hydrogel) on its outer surface to facilitate insertion/removal of the device into/from the patient. In some embodiments, device 20 includes one or more drugs (such as a muscle relaxant) on its outer surface. Device 20 can include various markers (such as radio-opaque (e.g., platinum) bands and/or MRI contrast agent(s)) to help the surgeon track or monitor the device.

Device 20 can be manufactured using conventional techniques. For example, balloons 24, 28 can be blow molded in a cavity similar to how angioplasty balloons are manufactured, and shaft 24 can be manufactured by extrusion. Balloons 24, 28 can be attached to shaft 24, for example, by an adhesive, heat bonding, sonic welding and/or laser bonding.

As indicated above, device 20 can be used in a variety of abdominal surgical procedures, such as, for example, bariatic surgery, fundoplication (e.g., partial or complete fundoplication), and other esophageal-related procedures, which are described below.

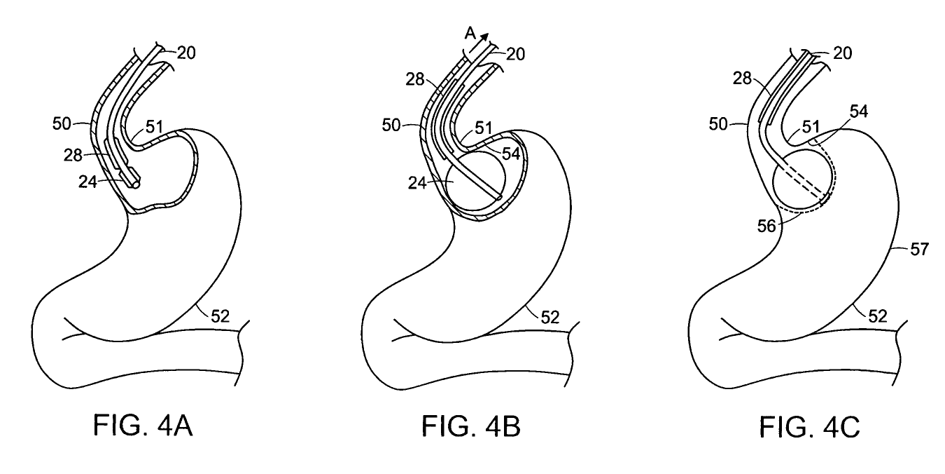

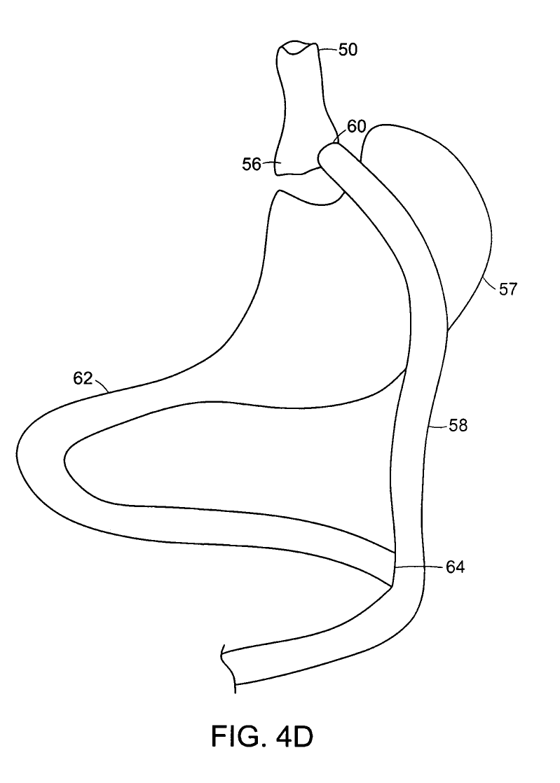

FIGS. 4A, 4B, 4C and 4D illustrate an example of a bariatic surgical procedure (as shown, a gastric bypass surgical procedure) using device 20. The procedure can be performed as a laparoscopic procedure or an open procedure. Referring to FIG. 4A, with balloons 24, 28 unexpanded, device 20 is introduced nasally or orally into the body, through the esophagus 50, past the GE junction 51, and into the stomach 52. The small size of device 20 allows it to be inserted through the cervical esophagus into the stomach easily, safely, and in a short amount of time (e.g., without having to slowly dilate the esophagus, thus reducing the risk of trauma such as tearing). In some embodiments, one or more instruments are passed through passageway 30 during the procedure. For example, an endoscope can be passed through passageway 30 to view the stomach 52 and/or to determine when distal end 26 has entered the stomach. The endoscope can also be passed through passageway 30 to view, inspect, and/or test the integrity of the completed surgical pouch or anastomosis. Other examples of instruments include an irrigation instrument, a suction instrument, a material (e.g., contrast agent) delivery instrument, and a light source (e.g., an optic fiber). The instrument(s) can be passed through passageway 30 any time during the surgical procedure as needed.

Referring to 4B, after first balloon 24 and/or second balloon 28 have traveled distally past the GE junction 51 and/or into the stomach 52, the first balloon 24 is expanded by introducing a fluid into proximal port 40, through passageway 32, and out of opening 38. The fluid can be, for example, a liquid (e.g., water, a saline solution, a chemiluminescent solution (such as luminal in an alkaline solution with hydrogen peroxide in the presence of iron or copper, or cyalume reacting with hydrogen peroxide in the presence of a dye)) and/or a gas (e.g., carbon dioxide). In some embodiments, first balloon 24 is formed of a non-compliant material such that, when fully expanded, the volume of the first balloon does not exceed a selected volume (e.g., approximately 30 cubic centimeters), regardless of any excessive amount of fluid that is introduced into the interior of the first balloon. In other embodiments, the expanded size of first balloon 24 can be adjusted by varying the amount of fluid introduced into the balloon. After first balloon 24 is expanded (e.g., fully expanded), device 20 is withdrawn proximally (arrow A), thereby contacting the first balloon against a proximal portion 54 of the stomach 52 and also locating the GE junction 51 (which is proximal to the proximal portion 54 of the stomach 52). During the withdrawal step, the surgeon may feel resistance to further withdrawal once first balloon 24 is adjacent the GE junction 51, thus providing an indication that the first balloon is properly positioned. In some embodiments, after locating the GE junction 51, second balloon 28 is expanded by introducing a fluid into proximal port 44, through passageway 34, and out of opening 42, for example, to maintain the patency of the esophagus 50.

Referring to FIG. 4C, after the expanded first balloon 24 contacts the proximal portion of the stomach 52, portions of the stomach are connected (e.g., stapled and/or sutured) together to form a small upper pouch 56. For example, using her hands (in an open surgical procedure) or the laparoscopic instruments, the surgeon can feel where the expanded first balloon 24 is in the stomach 52, and connect opposing wall portions of the stomach together around the first balloon to form upper pouch 56, which is subsequently separated from the remainder of the stomach, now a lower pouch 57. The expanded first balloon 24 allows the surgeon to form upper pouch 56 accurately and precisely, e.g., without having to estimate or to guess how big to make the upper pouch. Also, by limiting the length of shaft 22 that extends distally beyond first balloon 24, the distal portion of the shaft does not interfere with formation of upper pouch 56, and the final size of the upper pouch can match well with the size of the expanded first balloon, further enhancing the accuracy and precision of the surgical procedure.

After upper pouch 56 is formed, device 20 is withdrawn from the patient, and the remaining steps of a conventional bariatic surgical procedure can be performed. For example, referring to FIG. 4D, in a Roux-en-Y gastric bypass procedure, a segment of the small intestine 58 is brought from the lower abdomen and joined with upper pouch 56 to form an end-to-end anastomosis 60 created through an opening. Segment of the small intestine 58 (called the “Roux loop”) carries food from upper pouch 56 to the remainder of the intestines, where the food is digested. The larger lower pouch 57 (or distal detached stomach) and the attached segment of duodenum 62 are then reconnected to form another anastomotic connection 64 to the Roux loop. It is at connection 64 that digestive juices from the larger lower pouch 57 (distal stomach), the duodenum, the pancreas, and the liver enter the intestines help digest the food. The small size of upper pouch 56 forces the patient to eat at a slower rate, restricts the volume of food intake, and satiates the patient much more quickly, thereby reducing the patient's caloric intake and providing durable weight loss and/or weight maintenance.

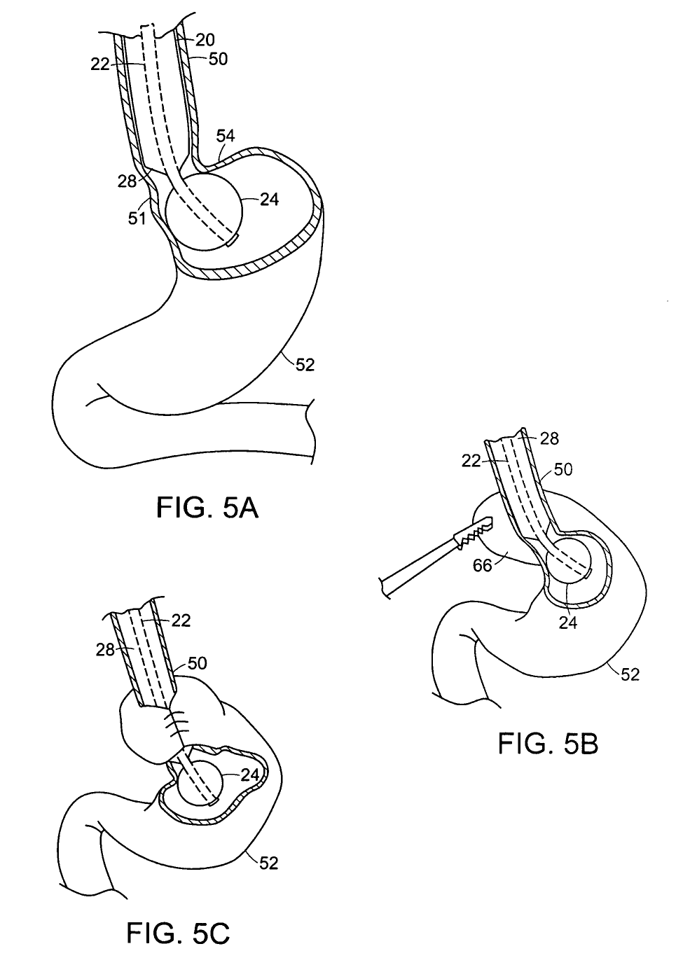

As indicated above, device 20 can also be used in other types of abdominal surgical procedures. FIGS. 5A, 5B, and 5C illustrate a complete fundoplication, which can be performed as an open or a laparoscopic procedure to treat gastroesophageal reflux disease (GERD) and hiatal or paraesophageal hernias. Referring to FIGS. 5A, device 20 is inserted through the esophagus 50 and into the stomach 52 as described above (FIG. 4A). After first balloon 24 is inserted past GE junction 51, the first balloon is expanded and withdrawn proximally to contact proximal portion 54 of the stomach 52 (FIG. 4B), thereby positioning second balloon 28 proximal to the GE junction. Subsequently, second balloon 28 is expanded to dilate the esophagus 50, to size the esophagus, and to maintain the patency of the esophagus so that the size and function of the esophagus are not compromised while the surgical repair and reconstruction are performed.

Referring to FIGS. 5B and 5C, an upper part 66 or fundus of the stomach 52 is then wrapped around the inferior part of the esophagus 50 to restore the function of the lower esophageal sphincter (which is defective in patients suffering from GERD), and sutured to the stomach. The expanded second balloon 28 in the esophagus 50 serves as a support or a temporary stent that prevents the wrapping of the stomach from being too tight. When complete, this procedure prevents reflux of gastric acid and/or the sliding of the fundus through an enlarged esophageal hiatus in the diaphragm. In other embodiments, the fundus is partially wrapped around the esophagus 50, such as in a Dor fundoplication or a Toupet fundoplication.

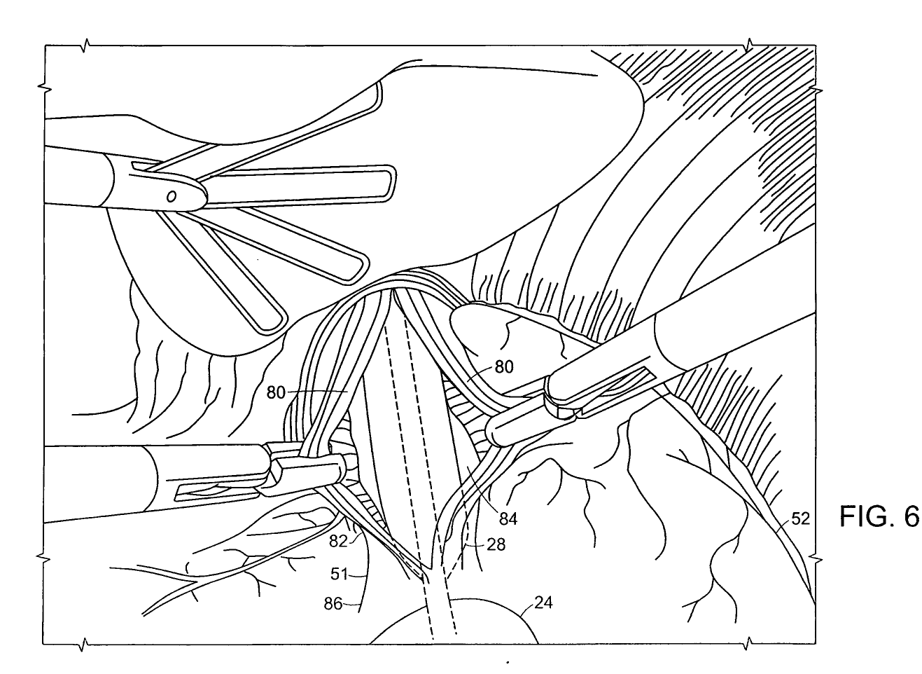

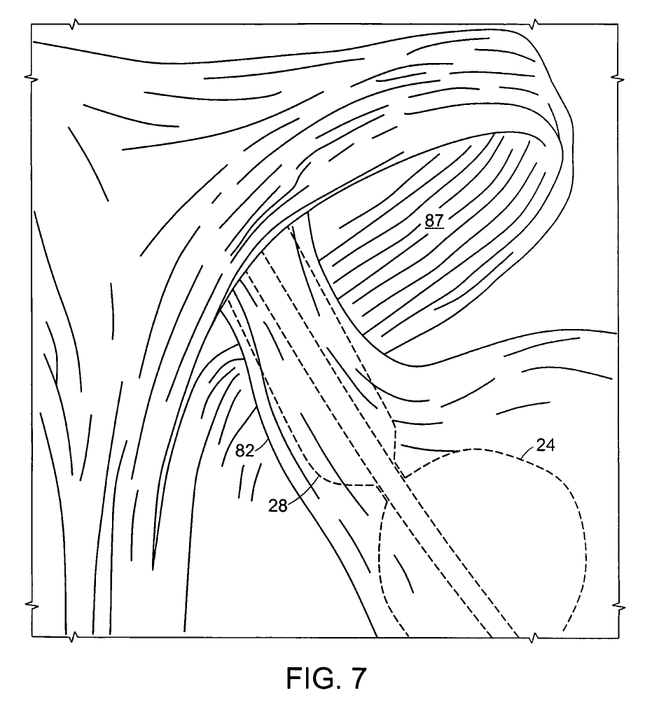

As another example, device 20 can be used in other esophageal-related surgical procedures, such as Heller myotomy, Hiatal and Paraesophageal hernia repairs (e.g., diaphragmatic hernia repairs), which can be performed as an open or a laparoscopic procedure. Referring again to FIG. 5A, in these esophageal related surgical procedures, device 20 is introduced into the body, first balloon 24 is expanded and positioned against proximal portion 54 of the stomach 52, and second balloon 28 is expanded in the esophagus 50 proximal to GE junction 51, as described above. Subsequent steps depend on the particular procedure being performed. Referring to FIG. 6, in a Heller myotomy, the thickened musculature 80 of the distal esophagus 82 is cut and divided down to underlying mucosa 84 longitudinally from approximately five centimeters above the GE junction 51 to approximately one centimeter onto the cardia 86 of the stomach 52. This allows food and liquids to pass into the stomach (e.g., to treat achalasia). Referring to FIG. 7, in a diaphragmatic hernia repair procedure, a defect in the diaphragm 87 can be closed or repaired around the distal esophagus 82. In some embodiments, sufficient space around the distal esophagus 82 is created to reduce or to avoid narrowing of stricture of the distal esophagus, which may result in difficulty swallowing. In some embodiments, the defect in the diaphragm can be repaired by not only closing or repairing the defect in the diaphragm, but a fundoplication or stomach wrap is added (e.g., FIGS. 5A-5C) to ensure success of the repair In these procedures, the expanded second balloon 28 in the esophagus 50 maintains the patency and size of the esophagus to prevent its collapse and help ensure that the repair of the diaphragm and the stomach wrap is not too tight, which can impede the function of the esophagus.

While a number of embodiments have been described, the invention is not so limited.

For example, device 20 can include one or more endoesophageal monitoring devices, such as a vitals monitor (e.g., for heart rate, and/or a core temperature) and an esophageal monitor. The monitoring device(s) can be incorporated in or on shaft 22.

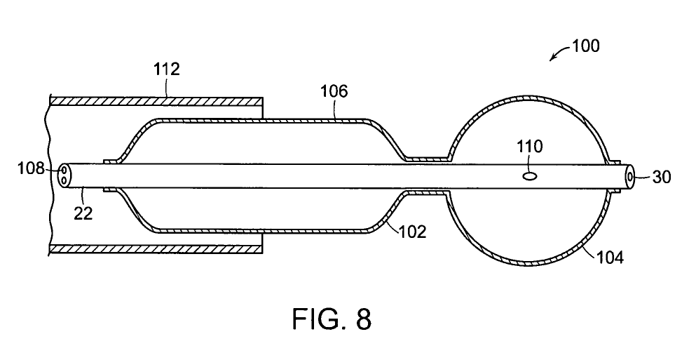

While device 20 is described above as having two spaced balloons, in other embodiments, the device includes one balloon having multiple lobes. Referring to FIG. 8, device 100 includes shaft 22 carrying a balloon 102 having a first expandable lobe 104 and a second expandable lobe 106. A fluid can be delivered into the interior of lobes 104, 106 through a passageway 108 and an opening 110 to expand the lobes, similar to passageway 32 and opening 38. Lobes 104, 106 are dimensioned and arranged similarly to first and second balloons 24, 28, respectively. Device 100 further includes a hollow sheath 112 configured to receive shaft 22 and balloon 102.

In use, device 100 is delivered into the stomach with sheath 112 positioned over lobes 104, 106. When first lobe 104 is positioned distal of the GE junction, sheath 112 is retracted proximally to expose the first lobe but to still extend over second lobe 106. Fluid is then introduced to expand first lobe 104. Sheath 112 prevents second lobe 106 from expanding substantially. Subsequently, the expanded first lobe 104 can be moved proximally to contact the proximal portion of the stomach and to locate second lobe 106 proximal of the GE junction. In some embodiments, formation of an upper pouch and steps of a bariatic surgical procedure can be performed. In other embodiments, sheath 112 can be fully retracted to expose second lobe 106, and with additional introduction of fluid, the second lobe can be expanded in the esophagus proximal to the GE junction, similar to second balloon 28 described above. The selected surgical procedure(s) can then be performed as described above.

All references, such as patents, patent applications, and publications, referred to above are incorporated by reference in their entirety.

Still other embodiments are within the scope of the following claims.

What is claimed is:

1. A method, comprising:
expanding a first balloon distally of a gastro-esophageal junction; and
contacting the first balloon to a proximal portion of a stomach.

2. The method of claim 1, further comprising connecting wall portions of the stomach together around a portion of the expanded first balloon.

3. The method of claim 1, further comprising expanding a second balloon proximally of the proximal portion of the stomach.

4. The method of claim 3, further comprising surgically reducing reflux from the stomach.

5. The method of claim 3, further comprising reducing or repairing a diaphragmatic defect.

6. The method of claim 3, further comprising performing at least one step of a Heller myotomy procedure.

7. The method of claim 3, wherein the second balloon is capable of having an expanded length of approximately 10 to approximately 15 centimeters.

8. The method of claim 3, wherein the second balloon is capable of having a maximum expanded outer diameter of approximately 20 millimeters.

9. The method of claim 1, further comprising performing at least one additional step of a bariatic surgical procedure.

10. The method of claim 1, further comprising orally or nasally introducing the first balloon into the body.

11. The method of claim 1, wherein the first balloon is capable of having an expanded volume of approximately 20 to approximately 30 cubic centimeters.

12. The method of claim 1, wherein the first balloon is carried by a shaft having a diameter of approximately 12 to approximately 28 French.

13. The method of claim 1, wherein the first balloon is carried by a shaft having a passageway extending along a longitudinal axis, and further comprising passing an instrument through the passageway, the instrument being selected from the group consisting of a pumping instrument, an irrigation instrument, a contrast agent delivery instrument, an endoscope, and a light source.

14. A method, comprising:
expanding a first balloon in a stomach; and
connecting wall portions of the stomach together around a portion of the expanded first balloon.

15. The method of claim 14, further comprising contacting the expanded first balloon to a proximal portion of the stomach.

16. The method of claim 14, further comprising expanding a second balloon proximally of the stomach.

17. The method of claim 14, further comprising orally or nasally introducing the first balloon into the body.

18. The method of claim 14, wherein the first balloon is capable of having an expanded volume of approximately 20 to approximately 30 cubic centimeters.

19. The method of claim 14, wherein the first balloon is capable of having a substantially spherical expanded volume.

20. The method of claim 14, wherein the first balloon is carried by a shaft having an outer diameter of approximately 24 to approximately 28 French.

21. The method of claim 14, further comprising performing at least one additional step of a bariatic surgical procedure.
